(19) United States
(12) Patent Application Publication
(10) Pub. No.: US 2012/0030969 A1
(43) Pub. Date: Feb. 9, 2012

(54) INSOLE FOR A SHOE HAVING A MOVABLE MASSAGING MEMBER

(76) Inventor: Sung Jo Lim, Busan (KR)

(21) Appl. No.: 13/263,332

(22) PCT Filed: Apr. 7, 2010
(86) PCT No.: PCT/KR10/02135

§ 371 Date: Oct. 6, 2011

(30) Foreign Application Priority Data
Apr. 7, 2009 (KR) 10-2009-0029685
May 12, 2009 (KR) 10-2009-0041132
Mar. 3, 2010 (KR) 10-2010-0018910
Apr. 7, 2010 (KR) 10-2010-0031772

(51) Int. Cl. A61F 5/14

(52) U.S. Cl. 36/141

(57) ABSTRACT

The present invention relates to a shoe, and more particularly, to an insole for a shoe having a movable massaging member comprising: an insole positioned underneath the sole of the foot; a metallic or magnetic securing body joined to the insole, across a partial space in the lengthwise direction; a massaging member disposed on the upper or lower surface of the insole; and a metallic or magnetic moving body which is joined to the massaging member and is magnetically drawn to the securing body, such that the massaging member is mobile.

### TECHNICAL FIELD

The present invention relates to a shoe, and more particularly, to a shoe having a massaging member which is configured to ensure a movement of a position of the massaging member and supports the sole of a user in a suitable position according to a shape of the sole of the user to thereby improve the massaging effect, encourage a correct pose of body and maximize the circulation of the blood by magnet.

### BACKGROUND ART

Generally, feet are known as the second heart and many people are concerned about the health of the foot. In recent years, shoes with various functions have been released in the market. Among those functional shoes, there are shoes for massaging the sole of the foot to obtain the effect of improving the circulation of the blood, etc.

Such shoes have projections formed in the sole member contacting the sole of the foot or protruding parts supporting the sole of the foot and fixed to the sole for massaging and supporting the sole, or have projections moving simply up and down. Thus, the shoes have a simple configuration for continuously pressing the sole of the foot and do not have a significant effect of blocking shock to the body. Further, as the projections do not uniformly conform to the sole of the foot, such shoes cause pain and body imbalance.

That is, as the projections are formed in the position not in line with the depth and position of the center of the sole of the foot of a user, a user may receive higher pressure and feel painful by continuous pressure upon putting on the shoes and walking. Further, as the fixed projections doe not conform to the structure of the feet of a user, the pose of the body of a user becomes twisted and the shoes do not absorb the shock and a user may feel tired easily.

### DISCLOSURE

### Technical Problem

The present invention has been made to solve the problems and it is an object of the present invention to provide a sole member for a shoe which comprises a movable massaging member so that a user adjusts a position of the massaging member voluntarily.

Also, it is another object of the present invention to provide a sole member for a shoe which comprises a massaging member that is adjusted in height and which makes the blood circulate more freely.

### Technical Solution

In order to achieve the object of the present invention, a sole member for a shoe having a movable massaging member comprises a sole member which is positioned underneath the sole of the foot; a metallic or magnetic securing body which is joined to the sole member across a partial space in a lengthwise direction; a massaging member which is disposed on an upper or lower surface of the sole member; and a metallic or magnetic moving body which is joined to the massaging member and is magnetically drawn to the securing body, such that the massaging member is mobile.

The massaging member comprises one of a hard material, an elastic material and an air-filled material.

The massaging member and the moving body are joined to each other by a bolt and a nut to adjust a height of the massaging member.

The massaging member comprises a first member comprising one of an air-filled material, a hard material and an elastic material, and a second member which is joined to a lower surface of the first member, comprises one side that is open, is hollow therein and discharges air when pressed.

The massaging member comprises a flange which protrudes from a circumference of the massaging member and is held by a guide opening formed in a guide plate joined to the sole member.

The massaging member and the moving body are joined to each other by a cushion body.

The sole member comprises a depressed or perforated part within which the moving body moves.

The sole member comprises a depressed part within which the moving body moves, and the securing body is installed in the depressed part and at least one cushion body is joined to a lower surface of the securing body.

In order to achieve the object of the present invention, a sole member for a shoe having a movable massaging member comprises a sole member positioned underneath the sole of the foot; a guide block which is formed in the sole member across a partial space in a lengthwise direction; a plurality of fitting grooves which is formed in the guide block; and a massaging member which comprises a fitting bar formed in a lower surface thereof to move a position.

### Advantageous Effect

As described above, a sole member for a shoe according to the present invention includes a movable massaging member for a user to properly adjust a position and height of the massaging member and change a degree of pressure when walking. Thus, the sole member for a shoe may reduce pain which may be caused by excessive pressure and support the sole of the foot in a required position to thereby correct a pose of the body, make the blood circulate more freely by magnet and help a user to maintain health.

Further, the sole member for a shoe according to the present invention allows air to flow within the shoe, reduces the smell of the foot and prevents propagation of bacteria.

### BRIEF DESCRIPTION OF DRAWINGS

FIG. 1 is an exploded perspective view of main parts of a sole member for a shoe according to a first exemplary embodiment of the present invention.

FIG. 2 illustrates main parts of the sole member for a shoe in an assembly state.

FIG. 3 illustrates the sole member joined to the uppers of the shoe.

FIG. 4 is a sectional view of a sole member for a shoe according to a second exemplary embodiment of the present invention.

FIG. 5 is a sectional view of a sole member for a shoe according to a third exemplary embodiment of the present invention.

FIG. 6 is a sectional view of a sole member for a shoe according to a fourth exemplary embodiment of the present invention.

FIG. 7 illustrates an example of the sole member for a shoe according to the fourth exemplary embodiment of the present invention which is employed in women's pumps.

FIG. 8 is a partial perspective view of a sole member for a shoe according to a fifth exemplary embodiment of the present invention.

FIG. 9 illustrates an example of a sole member for a shoe according to a sixth exemplary embodiment of the present invention.

FIG. 10 illustrates an example of a sole member for a shoe according to a seventh exemplary embodiment of the present invention.

FIG. 11 is a perspective view of a sole member for a shoe according to an eighth exemplary embodiment of the present invention.

### BEST MODE

Hereinafter, a sole member for a shoe having a movable massaging member according to the present invention will be described in detail with reference to accompanying drawings to help understand the technical spirit of the present invention. However, the drawings are provided to illustrate a preferable exemplary embodiment and it should be understood that other variants of the exemplary embodiment such as a simple structural modification by a third party are also included in the technical scope of the present invention.

In explaining the present invention, a sole member for a shoe may be interpreted variously depending on the type of a shoe. The sole member for a shoe may mean an insole as a representative example or a thing like a nonwoven positioned below the insole and joined to the uppers of the shoe or a texon,—a midsole, or an upper surface exposed to the outside in a shoe such as a sandal.

A sole member for a shoe which includes a movable massaging member according to the present invention includes a sole member 100 which is positioned underneath the sole of the foot, a metallic or magnetic securing body 200 joined to the sole member 100 across a partial space in a lengthwise direction, a massaging member 300 disposed on an upper or lower surface of the sole member 100, and a metallic or magnetic moving body 400 joined to the massaging member 300 and is magnetically drawn to the securing body 200.

As the moving body 400 joined to the massaging member 300 may be controlled by the securing body 200 and the magnetic force or may change the position of the massaging member 300 as necessary, a shoe which is put on by a user mostly comfortably according to a user's body condition is provided.

### First Exemplary Embodiment

The sole member 100 according to the present invention may vary depending on a type of a shoe as described above. The sole member 100 according to a first exemplary embodiment of the present invention relates to a sole member suitable for snickers.

That is, the sole member 100 which is coupled to the uppers 10 of the shoe along a circumference of the uppers 10 by sewing includes a nonwoven.

When a shoe is manufactured, the insole is provided on the sole member 100 including the nonwoven, and the midsole is joined to a lower part of the sole member 100.

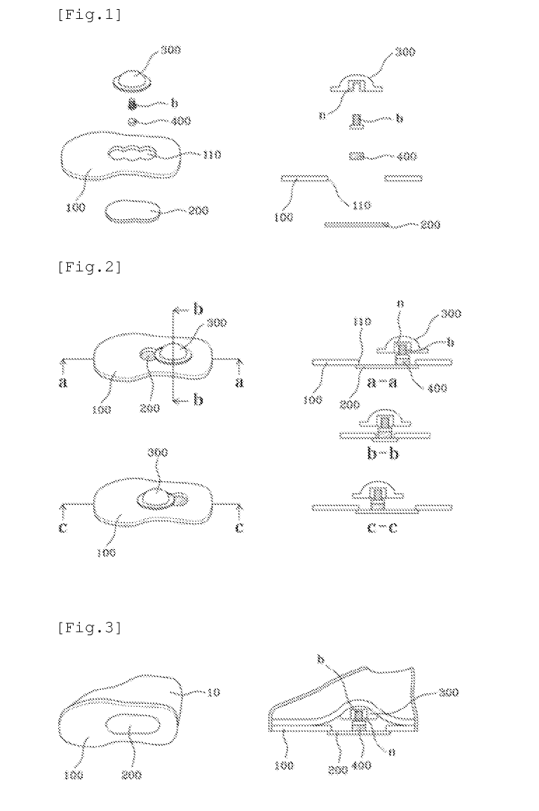

FIG. 1 is an exploded perspective view of main parts of the sole member for a shoe according to the first exemplary embodiment of the present invention. FIG. 2 illustrates main parts of the sole member for a shoe in an assembly state. FIG. 3 illustrates the sole member 100 joined to the uppers 10 of the shoe.

The sole member 100 is joined to the securing body 200, and preferably, the securing body 200 is positioned in an area falling under the arch-shaped part of the sole of the foot. The securing body 200 may include a metallic or magnetic plate. The massaging member 300 is disposed on an upper or lower surface of the sole member 100 and joined to the moving body 400. The moving body 400 is metallic or magnetic and may be controlled by the securing body 200 and the magnetic force.

If the securing body 200 includes a metallic material, the moving body 400 includes a magnet. If the securing body 200 includes a magnet, the moving body 400 includes a metallic material. The massaging member 300 may be disposed on the upper or lower surface of the sole member 100, which depends on the installation position of the securing body 200.

That is, according to the present exemplary embodiment, the metallic, plate-shaped securing body 200 is attached to the lower surface of the sole member 100 including the nonwoven. The massaging member 300 is provided on an upper surface of the sole member 100.

Further, the sole member 100 includes a depressed or perforated part to limit a moving area of the moving body 400, which is called a limitation groove 110. The moving body 400 which is joined to the massaging member 300 is inserted into the limitation groove 110. The limitation groove 110 may be shaped like a straight line or may be uneven to prevent a flow.

The massaging member 300 is preferably shaped like a dome, and may include a hard material, an elastic material such as a foaming agent or an air-filled material.

More preferably, a bolt b and a nut n may be interposed between the massaging member 300 and the moving body 400 for a user to adjust a height of the massaging member 300. That is, the nut n is built in the lower surface of the massaging member 300 and the bolt b is connected to the upper surface of the moving body 400 so that a user adjusts the height of the massaging member 300 by the bolt and nut.

### Second Exemplary Embodiment

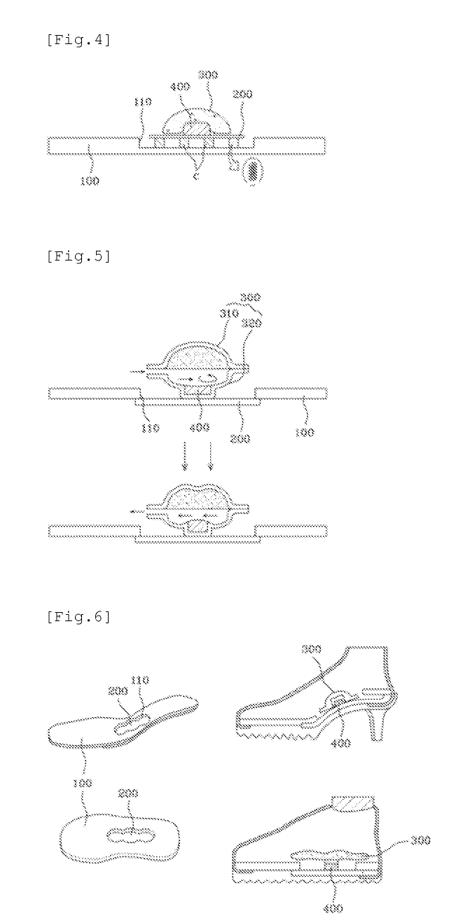

Hereinafter, a sole member for a shoe according to a second exemplary embodiment of the present invention will be described. FIG. 4 is a sectional view of the sole member for a shoe according to the second exemplary embodiment of the present invention.

As shown therein, a sole member 100 according to the second exemplary embodiment includes a limitation groove 110 which is depressed from the sole member 100. The metallic, plate-shaped securing body 200 is installed in the limitation groove 110. Particularly, at least one cushion body c is joined to a lower surface of the securing body 200 and is fixed to the sole member 100. The cushion body c may include a plurality of springs or a plurality of foaming columns.

The massaging member 300 is disposed on an upper surface of the securing body 200 and is joined to the magnetic moving body 400 to move the position of the massaging member 300 back and forth.

### Third Exemplary Embodiment

A sole member for a shoe having a movable massaging member according to the third exemplary embodiment of the present invention includes a configuration as shown in FIG. 5. Particularly, the massaging member 300 includes a first member 310 and a second member 320.

Basically, the securing body 200 is joined to the sole member 100, but joined to the lower surface of the sole member 100, leaving the limitation groove 110 as a depressed part therebetween. The first member 310 forming an upper part of the massaging member 300, the second member 320 which is joined to the lower surface of the first member 310 and the moving body 400 formed in the second member 320 are provided.

The first member 310 may be shaped like a dome, and may include one of a hard material, an elastic material and an air-filled material. The second member 320 has one side open to discharge air when pressed. The second member 320 is hollow therein. The second member 320 includes an elastic material, and is compressed and discharges air when a load is applied thereto by a user's walking and is restored to its original state when the load is removed.

With the foregoing configuration according to the present exemplary embodiment, air is discharged from the second member 320 whenever a user walks and the sole of the foot may be maintained comfortably.

### Fourth Exemplary Embodiment

Hereinafter, a sole member for a shoe according to a fourth exemplary embodiment of the present invention will be described. FIG. 6 illustrates the sole member for a shoe according to the fourth exemplary embodiment of the present invention. According to the present exemplary embodiment, the sole member 100 includes a texon employed in a high heel or shoes.

The sole member 100 includes a texon and the securing body 200 includes a metallic plate body. The securing body 200 is provided within the texon, and a limitation groove 110 is depressed in a position falling under the arch-shaped part of the sole of the foot. The securing body 200 is positioned in the bottom of the limitation groove 110, and the limitation groove 110 is preferably uneven. The moving body 400 is joined to the lower surface of the massaging member 300, which is shaped like a dome, etc.

The massaging member 300 may include one of a hard material, an elastic material and an air-filled material.

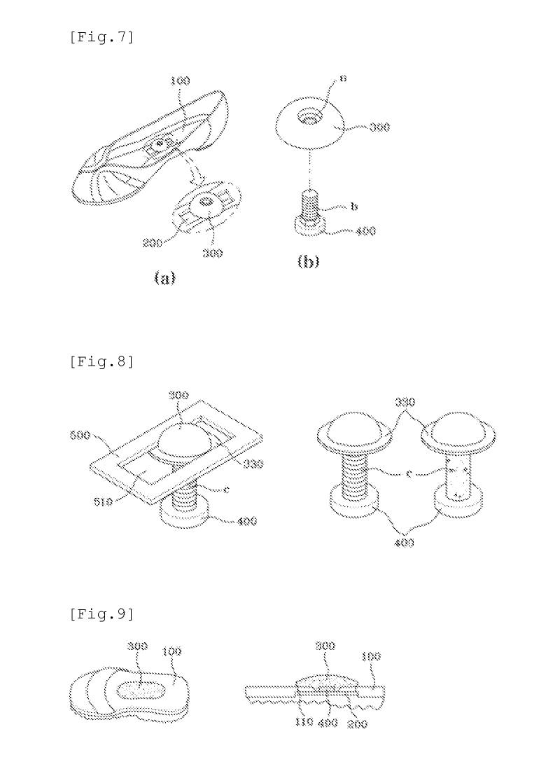

FIG. 7 illustrates the sole member 100 according to the fourth exemplary embodiment of the present invention which is employed in women's pumps. The massaging member 300 and the moving body 400 are joined to each other by a bolt b and a nut n to adjust a height of the massaging member 300.

### Fifth Exemplary Embodiment

Hereinafter, a sole member for a shoe according to a fifth exemplary embodiment of the present invention will be described. FIG. 8 is a partial perspective view of the sole member for a shoe according to the fifth exemplary embodiment of the present invention.

Basically, a securing body (not shown) is joined to a sole member (not shown). The moving body 400 is joined to the massaging member 300. A flange 330 protrudes from a circumference of the massaging member 300, and a guide plate 500 having a guide opening 510 formed therein is joined to the sole member so that the flange 330 is held by a lower part of the guide plate 500. The massaging member 300 may move along the guide opening 510 and the flange 330 prevents the guide plate 500 from being separated therefrom to improve stability.

More preferably, the massaging member 300 and the moving body 400 are connected to each other by a cushion body c such as a spring or a foaming agent so that the massaging member 300 may move up and down and provide the massaging effect upon receiving the load.

The sole member 100 may be the same as those according to the foregoing exemplary embodiments, or insoles or midsoles may be used as the sole member 100.

### Sixth Exemplary Embodiment

FIG. 9 illustrates a sole member for a shoe according to a sixth exemplary embodiment of the present invention which is employed in a sandal.

As shown therein, a sole member 100 which contacts the sole of the foot is exposed to the outside. The movable massaging member 300 is installed in an upper surface of the sole member 100. That is, the limitation groove 110 is depressed from the sole member 100, and the securing body 200 is installed in the sole member 100. The moving body 400 is formed in the lower surface of the massaging member 300 so that a user may adjust a position of the massaging member 300.

### Seventh Exemplary Embodiment

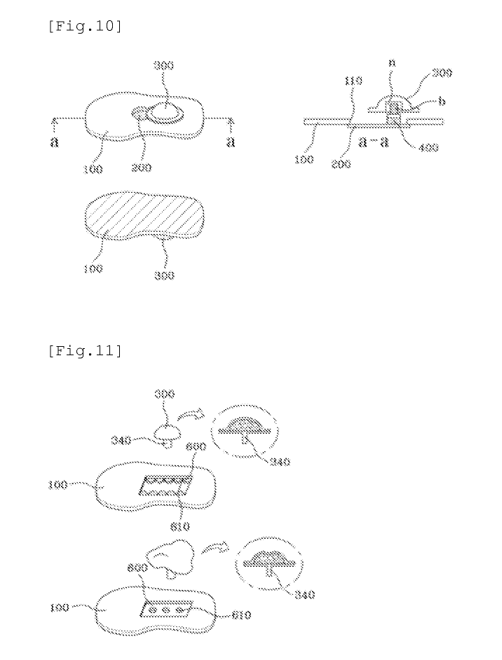

A sole member for a shoe according to a seventh exemplary embodiment of the present invention includes an insole for a shoe. FIG. 10 illustrates an example of the sole member for a shoe according to the present exemplary embodiment.

As shown therein, the massaging member 300 is installed in a center of the insole to support the arch-shaped part of the sole of the foot. The securing body 200 is attached to a lower surface of the insole and the massaging member 300 is disposed on the upper surface of the sole member 100 so that the moving body 400 joined to the massaging member 300 is controlled by the securing body 200 and the magnetic force. A bolt and a nut may be provided between the massaging member 300 and the moving body 400 to adjust the height of the massaging member 300.

The massaging member 300 may be formed in a lower surface as well as in the upper surface of the insole 100. Preferably, a limitation groove 110 is depressed from the sole member 100 and the position of the massaging member 300 is moved within the limitation groove 110.

The massaging member 300 may be installed in a lower surface of the sole member 100 for movement.

### Eighth Exemplary Embodiment

Another type of a sole member for a shoe will be described. FIG. 11 is a perspective view of a sole member for a shoe according to an eighth exemplary embodiment of the present invention.

A sole member for a shoe according to the present exemplary embodiment includes a sole member 100, a guide block 600, a fitting groove 610 and a massaging member 300. As shown therein, the massaging member 300 may move its position with respect to the sole member 100. To enable the movement of the massaging member 300, the guide block 600 is partially formed in the sole member 100 in a lengthwise direction.

The guide block 600 is joined to a center of the sole member 100, and includes a plurality of fitting grooves 610. A fitting bar 340 which protrudes from a lower part of the massaging member 300 may be inserted into the fitting groove 610. The fitting groove 610 may be uneven or include a circular groove.

The guide block 600 is relatively hard and is fixed with the fitting bar 340 which is joined to the fitting groove 610.

As described above, the sole member for a shoe according to the present invention may be applicable to various shoes. The sole member may include an insole, nonwoven, midsole, a texon depending on the type of shoes. The sole member according to the foregoing exemplary embodiments may include one of those described above, but not limited to those provided as a specific example.

That is, the sole member which is described to include a texon or nonwoven may further include other insoles or midsoles.

### INDUSTRIAL APPLICABILITY

A sole member for a shoe having a movable massaging member according to the present invention may move a position of the massaging member and adjust a height of the massaging member according to a user, and improve the circulation of the blood by magnet and apply to various types of shoes with superb advantages.

1. A sole member for a shoe having a movable massaging member comprising:
a sole member which is positioned underneath the sole of the foot;
a metallic or magnetic securing body which is joined to the sole member across a partial space in a lengthwise direction;
a massaging member which is disposed on an upper or lower surface of the sole member; and
a metallic or magnetic moving body which is joined to the massaging member and is magnetically drawn to the securing body, such that the massaging member is mobile.

2. The sole member for a shoe according to claim 1, wherein the massaging member comprises one of a hard material, an elastic material and an air-filled material.

3. The sole member for a shoe according to claim 2, wherein the massaging member and the moving body are joined to each other by a bolt and a nut to adjust a height of the massaging member.

4. The sole member for a shoe according to claim 1, wherein the massaging member comprises a first member comprising one of an air-filled material, a hard material and an elastic material, and a second member which is joined to a lower surface of the first member, comprises one side that is open, is hollow therein and discharges air when pressed.

5. The sole member for a shoe according to claim 1, wherein the massaging member comprises a flange which protrudes from a circumference of the massaging member and is held by a guide opening formed in a guide plate joined to the sole member.

6. The sole member for a shoe according to claim 5, wherein the massaging member and the moving body are joined to each other by a cushion body.

7. The sole member for a shoe according to claim 1, wherein the sole member comprises a depressed or perforated part within which the moving body moves.

8. The sole member for a shoe according to claim 1, wherein the sole member comprises a depressed part within which the moving body moves, and the securing body is installed in the depressed part and at least one cushion body is joined to a lower surface of the securing body.

9. A sole member for a shoe having a movable massaging member comprising:
a sole member positioned underneath the sole of the foot;
a guide block which is formed in the sole member across a partial space in a lengthwise direction;
a plurality of fitting grooves which is formed in the guide block; and
a massaging member which comprises a fitting bar formed in a lower surface thereof to move a position.

10. The sole member for a shoe according to claim 2, wherein the sole member comprises a depressed or perforated part within which the moving body moves.

11. The sole member for a shoe according to claim 3, wherein the sole member comprises a depressed or perforated part within which the moving body moves.

12. The sole member for a shoe according to claim 4, wherein the sole member comprises a depressed or perforated part within which the moving body moves.

13. The sole member for a shoe according to claim 5, wherein the sole member comprises a depressed or perforated part within which the moving body moves.

14. The sole member for a shoe according to claim 6, wherein the sole member comprises a depressed or perforated part within which the moving body moves.

15. The sole member for a shoe according to claim 2, wherein the sole member comprises a depressed part within which the moving body moves, and the securing body is installed in the depressed part and at least one cushion body is joined to a lower surface of the securing body.

16. The sole member for a shoe according to claim 3, wherein the sole member comprises a depressed part within which the moving body moves, and the securing body is installed in the depressed part and at least one cushion body is joined to a lower surface of the securing body.

17. The sole member for a shoe according to claim 4, wherein the sole member comprises a depressed part within which the moving body moves, and the securing body is installed in the depressed part and at least one cushion body is joined to a lower surface of the securing body.

18. The sole member for a shoe according to claim 5, wherein the sole member comprises a depressed part within which the moving body moves, and the securing body is installed in the depressed part and at least one cushion body is joined to a lower surface of the securing body.
